(19) United States
(12) Patent Application Publication
(10) Pub. No.: US 2013/0023751 A1
(43) Pub. Date: Jan. 24, 2013

(54) Water retention monitoring

(71) Applicants: Samuel Victor Lichtenstein, Vancouver (CA); Daniel Gelbart, Vancouver (CA)

(72) Inventors: Samuel Victor Lichtenstein, Vancouver (CA); Daniel Gelbart, Vancouver (CA)

(21) Appl. No.: 13/135,867

(22) Filed: Jul. 18, 2011

(51) Int. Cl. A61B 5/053

(52) U.S. Cl. 600/390; 600/547

(57) ABSTRACT

The water contents of the tissue is measured by placing part of the body, such as the arm or ankle, between two capacitive electrodes and calculating the water contents based on the dielectric properties of the tissue. The device is shaped like a bracelet or hinged clip. When placed over part of the body the hinge position is measured to normalize the reading for the tissue thickness. The device can alert the user of water retention, and can also contact the physician directly via a wireless link.

### FIELD OF THE INVENTION

The invention is in the medical field, and more specifically cardiac medicine.

### BACKGROUND OF THE INVENTION

It is well known in medicine that as the heart starts failing the water retention in body tissues goes up. It was previously shown that monitoring this water retention is a good diagnostic tool for predicting heart failure. Heart failure may take a long time to develop and the cost of monitoring the patients, in order to determine when intervention is needed, is high. The advantage of monitoring the status of the cardiac system by water retention in the body is the ability to perform the test at home, without help from medical personnel. Previous attempts to use at-home monitoring were mainly based on the weight of the patient. Such a method is inaccurate as rapid weight gain (from overeating) will have the same symptoms as water retention and weight loss can mask the effect of water retention. It is desired to have a low cost and simple to use system that measures the water contents of the body directly, without being affected by the shape or weight of the body and without requiring calibration to a specific person.

Prior art devices for measuring water retention by electrical methods are based on impedance measurement, single sided capacitance measurements or optical measurement. Impedance methods require good electrical contact with the skin, using a special paste, similar to EKG electrodes, and are not suitable for home use. Single sided (i.e. both electrodes on the same side of the tissue) capacitance measurements, such as US patent application 20050177061, are inaccurate as the electric field single sided electrodes generate is non-uniform. For example if a fatty tissue is near the skin and a muscle is below the fatty tissue the reading will be different than when the muscle is above the fatty tissue. In a more uniform electric field, as created by a parallel plate capacitor, the order of the layers will not affect the reading.

Single sided optical measurements, or even through-the tissue optical measurement are hard to perform accurately since the optical properties do not change much with the water contents. This can be seen in FIGS. 8A, 8B and 8C of US patent application 20080220512. One object of the invention is to create a low cost and simple device that can be used by non-medical personnel, even at home, and will not require calibration for a specific body location or person. A further object is to make the measurement unaffected by skin resistance, by using the capacitive part of the impedance rather the resistive part. Since water has a very high dielectric constant (over 80) compared to other tissue components, the dielectric constant mainly reflects the water contents of the tissue.

### SUMMARY OF THE INVENTION

The water contents of the tissue is measured by placing part of the body, such as the arm or ankle, between two capacitive electrodes and calculating the water contents based on the dielectric properties of the tissue. The device is shaped like a bracelet or hinged clip. When placed over part of the body the hinge position is measured to normalize the reading for the tissue thickness. The device can alert the user of water retention, and can also contact the physician directly via a wireless link.

### DESCRIPTION OF THE DRAWINGS

FIG. 1 is an isometric phantom view of the device.

FIG. 2 is a schematic diagram of the electronic circuit.

FIG. 3 shows the change in the dielectric constant of tissue as a function of the water contents.

FIG. 4 is an isometric phantom view of the device using a different thickness sensing method.

FIG. 5 is a device in the shape of a bracelet.

### DESCRIPTION OF THE PREFERRED EMBODIMENT

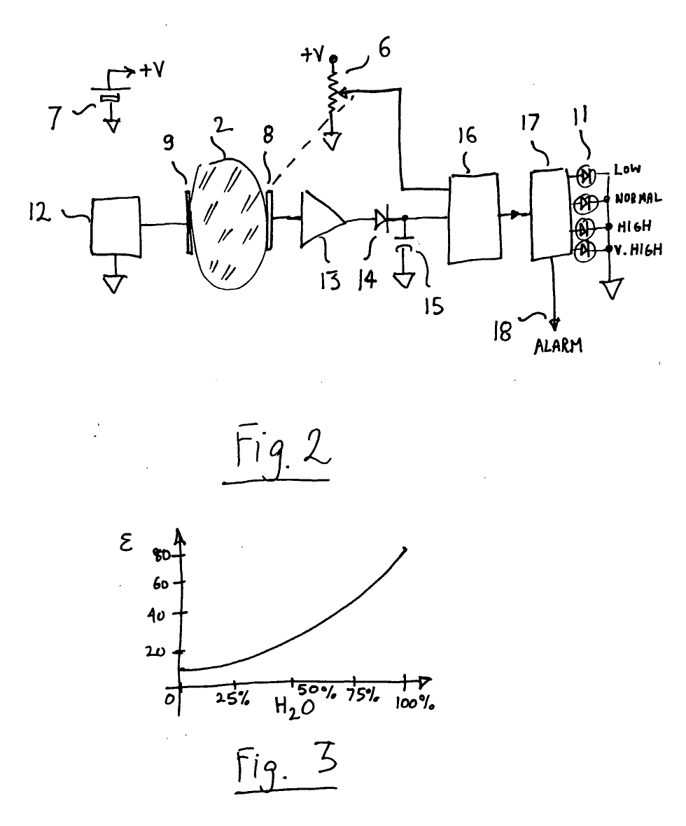

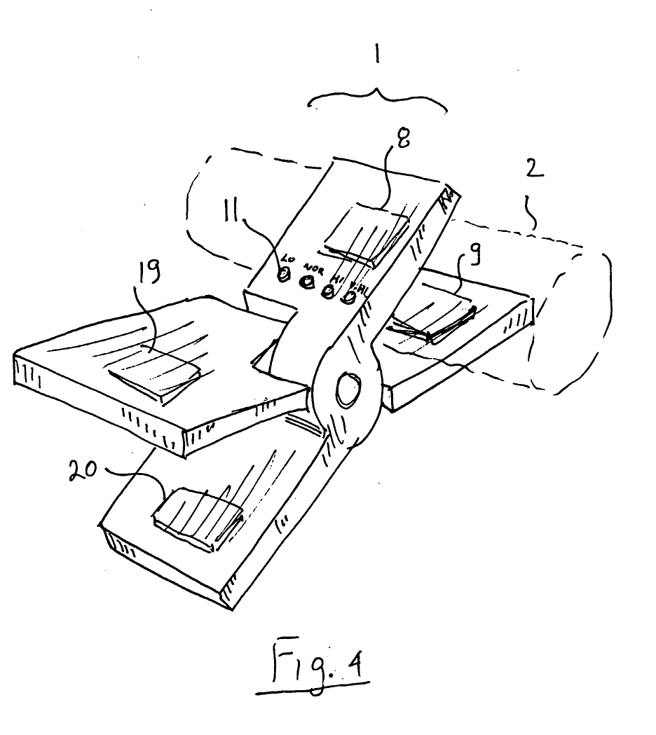

It is well known that the electrical properties of tissue are affected by the water contents. A suitable electrical property for monitoring water contents is the dielectric constant, also known as permittivity. The measurement can be done at a wide range of frequencies, from KHz to GHz. The range of 1 MHz to 100 MHz is particularly useful because of the ease of implementation, and in particular frequencies that fall in the unregulated ISM band, such as 6.78 MHz, 13.5 MHz or 27 MHz are convenient to use. The capacitance is measured by placing the tissue between two electrodes. The higher the water contents the higher the capacitance. The approximate dependence of the dielectric constant of tissue on the water contents is shown in FIG. 3. Preferably the electrodes are covered by a thin (typically 0.01 mm-0.1 mm) layer of electrical insulation in order to have only capacitive currents and block resistive currents. The dielectric properties of the insulation are not important as it is very thin compared to the measured tissue. Since the capacitance is also affected by the tissue thickness, following the formula C=εA/d (where ε is the dielectric constant, A is the electrode area and d is the tissue thickness), the reading has to be compensated for the tissue thickness. This can be done in several ways, such as incorporating a sensing element to sense the thickness of the tissue in the device.

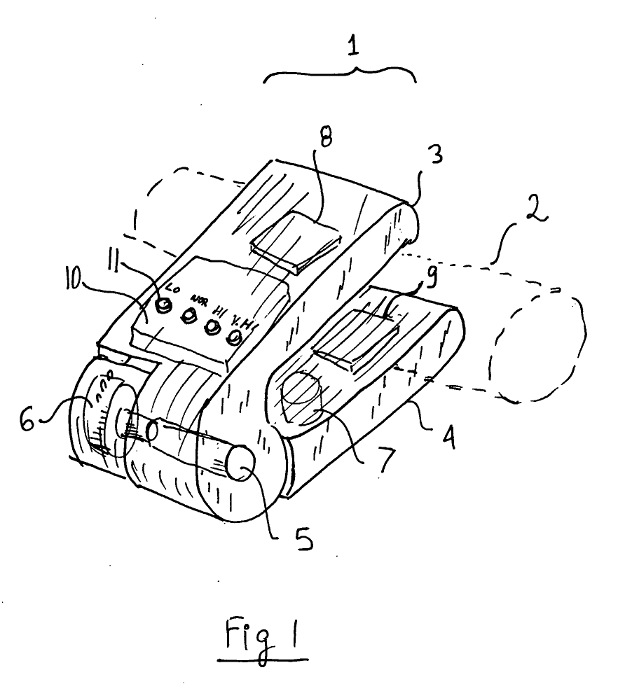

Referring now to FIG. 1, a device 1 is placed over body tissue 2 (such as arm). The device comprises of parts 3 and 4 coupled by hinge 5. A variable resistor 6 measured the rotation of hinge 5 and, indirectly, the tissue thickness. Arms 3 and 4 incorporate electrodes 8 and 9, battery 7, electronics module 10, and readout 11. Readout 11 can be a visible indicator, such as LEDs. Battery life will be very long as the device needs to be turned on for about one second once per day, this even a watch type battery will last many years. Assuming power consumption of 0.3 W, a 3V/100 mAH lithium watch battery will last about 10 years.

Electrodes 8 and 9 can be made to tilt in one or two planes in order to better fit the body part on which they are placed. A small air gap, typically below 1 mm, will not introduce a large error but if the whole area of the pad is in contact with the skin accuracy is improved. Another option is to use shallow sealed bags filled with an electrically conductive liquid or gel as electrodes. Such bags will comply and fit well any body part. FIG. 2 shows typical electronics that can be used to implement the device. Oscillator 12 generates a frequency typically in the MHz range at amplitude of typically 1V. Electrodes 8 and 9 can be coated with a thin layer of electrical insulation, as explained earlier. This allows only the capacitive component of the current to pass. More elaborate water contents monitoring methods can be used, measuring the complex impedance of the tissue at one or more frequencies. Such systems supply more information on the type of tissue and its composition. And can provide more accurate water level contents measurements. The capacitive current is amplified by amplifier 13, detected by detector 14, filtered by capacitor 15 and fed to a normalization element 16. The normalization element 16 compensates the reading for the tissue thickness, to make the reading independent of thickness. In the simplest form the thickness is measured by variable resistor 6 sensing the hinge rotation. Since both the hinge rotation and the capacitance are not linear with thickness it is best to use a digital correction based on a look-up table or algorithm, although a simple analog multiplier can give reasonable accuracy. In such a case the capacitive current is simply multiplied by the thickness.

The normalized output is fed to readout unit 17 which turns on LEDs 11 as well as generating any required alarm signal 18. The alarm signal can be visible, audible, a wireless transmitter to automatically alert a physician via a mobile phone network or the Internet, or any combination of the above.

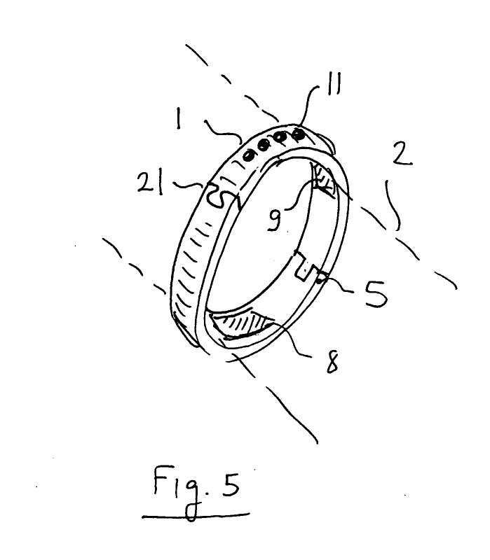

An alternate thickness compensation method is shown in FIG. 4. The clip has an extra pair of electrodes 19 and 20. The capacitive current through the tissue is compared to the current through the air gap between electrodes 19 and 20. The ratio of the currents is the dielectric constant of the tissue, from which the water contents is derived according to FIG. 3.

Sometimes accuracy can be improved by measuring the electrical impedance at multiple frequencies, for example 1 KHz, 100 KHz and 10 MHz. The value of the dielectric constant derived from these measurements will not be the same, as the dielectric constant, which has a real and imaginary part, is also a function of frequency. Each measurement forms an independent equation and the number of unknowns can equal the number of independent equation. Such change of electrical properties with frequency is known as dispersion and the art of measuring dispersion based on measurements at several frequencies is well known in electrical engineering. Dispersion can supply further information about the composition of the tissue. To measure dispersion oscillator 12 is set up to generate several frequencies, either sequentially or at the same time. Detector 14 measures the electrical signal at each one of those frequencies. More complete data about tissue discrimination using multiple frequencies is given in US patent application 2007/0270688, by the same inventors. This application is hereby incorporated by reference.

The device can be configured in other forms, for example in the form of a permanently worn bracelet having the two electrodes at diametrically opposed positions. The bracelet can be worn, for example, on the wrist or the ankle.

The electronic circuit can be designed to turn on for a very brief period, say one second, once per day. This will allow even a very small battery to last many years. Since the bracelet has a fixed size, thickness compensation can be eliminated by calibration. Such a design is shown in FIG. 5. The bracelet is locked in place by closure 21. Electrodes 8 and 9 can be spring loaded to assure good contact with tissue.

In operation the patient simply slips the device over their arm (or other body part) daily and sees the result instantly. There is minimal time delay involved in the measurement, typically under one second. In case of a bracelet, the patient simply wears the bracelet at all times.

An alternate method of detecting heart failure is to measure the change in electrical impedance rather than the actual impedance. Any rapidly decreasing impedance signifies water retention, as the impedance decreases as the dielectric constant, reflecting water content, is increasing. By setting up a baseline from daily measurements between two electrodes, a trend can be established without knowing the absolute value of the impedance. This eliminates the need to know the tissue thickness.

While the main application of the invention is monitoring of heart failure it can be used for other medical applications such as monitoring kidney function.

1. A device for monitoring heart failure by measuring the increase in water retention in the body tissue of the patient, said measuring performed by measuring the electrical properties of said tissue in a manner which compensates for tissue thickness.

2. A device for monitoring heart failure by measuring the increase in water retention in a patient's tissue, said device measuring the dielectric constant of the tissue by placing the tissue between two electrodes and compensating for the tissue thickness.

3. A device for monitoring heart failure by measuring the increase in water retention in a patient's tissue, said device sensing the change in the impedance of the tissue by placing the tissue between to electrodes and comparing the current impedance to a previously recorded impedance.

4. A device as in claim 1 wherein the electrical impedance is measured at multiple frequencies.

5. A device as in claim 2 wherein compensation for tissue thickness is performed by sensing the position of the electrodes.

6. A device as in claim 1 wherein the sensing is performed for a short duration, followed by turning the device off for a significantly longer duration in order to conserve electrical power.

7. A device as in claim 2 wherein the sensing is performed for a short duration, followed by turning the device off for a significantly longer duration in order to conserve electrical power.

8. A device as in claim 3 wherein the sensing is performed for a short duration, followed by turning the device off for a significantly longer duration in order to conserve electrical power.

9. A device as in claim 3 formed in the shape of a bracelet to be worn on the wrist or the ankle.

10. A device as in claim 2 wherein the electrodes incorporate sealed bags containing an electrically conductive liquid or gel.

11. A device as in claim 2 wherein the electrodes incorporate sealed bags containing an electrically conductive liquid or gel.

12. A device as in claim 1 wherein the patient is alerted to said water retention by a visible or audible signal.

13. A device as in claim 1 wherein the patient's doctor is alerted to said water retention by said device using a wireless link.

14. A device as in claim 2 wherein the patient is alerted to said water retention by a visible or audible signal.

15. A device as in claim 2 wherein the patient's doctor is alerted to said water retention by said device using a wireless link.

16. A device as in claim 3 wherein the patient is alerted to said water retention by a visible or audible signal.

17. A device as in claim 3 wherein the patient's doctor is alerted to said water retention by said device using a wireless link.
